(19) United States
(12) Patent Application Publication
(10) Pub. No.: US 2005/0136492 A1
(43) Pub. Date: Jun. 23, 2005

(54) Affinity labeling of enzymes for detection of enzyme activity level in living cells

(76) Inventors: David J. Phelps, Madison, WI (US); Gary L. Johnson, Bloomington, MN (US); Brian W. Lee, Minnetonka, MN (US); Zbigniew Darzynkiewicz, Chappaqua, NY (US); Jerzy Grabarek, Lodz (PL)

Correspondence Address: SCHWEGMAN, LUNDBERG, WOESSNER & KLUTH, P.A., P.O. BOX 2938, MINNEAPOLIS, MN, 55402, US

(21) Appl. No.: 10/872,818

(22) Filed: Jun. 21, 2004

Related U.S. Application Data
(63) Continuation of application No. PCT/US02/40722, filed on Dec. 19, 2002.
(60) Provisional application No. 60/342,778, filed on Dec. 21, 2001.

(51) Int. Cl. G01N 33/574

(52) U.S. Cl. 435/7.23

(57) ABSTRACT

The invention provides assay methods and reagents useful for evaluating the level of enzyme activities within living cells. Enzyme activity levels within living cells, such as caspases and Serine proteases, can be key determinates in assessing; 1) the apoptotic state of a cell, 2) the presence of tumor (cancer) cells, 3) the predictive efficacy of a chemotherapeutic treatment regimen using a particular therapeutic agent or process, 4) the probability of graft rejection or acceptance, identification of the up or down regulation relationships of serine proteases and caspases within living cell systems, provides a rapid, yet finely tuned mechanism for predicting the current and future state of these cell populations, and 5) the disease state status of a cell.

### RELATED APPLICATIONS

This application is a continuation under 35 U.S.C. 111(a) of International Application No. PCT/US02/40722 filed Dec. 19, 2002 and published in English as WO 03/058194 A2 on Jul. 17, 2003, which claimed priority under 35 U.S.C. 119(e) from U.S. Provisional Application Ser. No. 60/342,778 filed Dec. 21, 2001, which applications and publication are incorporated herein by reference.

### BACKGROUND OF THE INVENTION

Proteases are essential components in the cellular disassembly process that drives the programmed cell death mechanism called apoptosis. The involvement of cysteine proteases that specifically cleave peptides at the carboxyl side of aspartate residues (caspases) has been extensively studied (Alnemri et al., Cell, 1996, 87:171-173; Kaufmann et al., Cancer Res., 1993, 53:3976-3985; Lazebnik et al., Nature, 1994, 371:346-347; Budihardjo et al., Annu Rev Cell Dev Biol, 1999, 15:269-290; Earnshaw et al., Annu Rev Biochem, 1999, 68:383-424; Nicholson et al., Cell Death Differ, 1999, 6:1028-1042; Zhang et al., Cell Death Differ, 1999, 6:1043-1053; Stennicke et al., Cell Death Differ, 1999, 6:1060-1066).

Compared to caspases, participation of proteases in the cell's demise by apoptosis, is less understood (Johnson et al., Leukemia, 2000, 14:1695-1703). One group of proteases is the serine (Ser) proteases. These enzymes contain Ser at the active center, which participates in the formation of an intermediate ester to transiently form an acyl-enzyme complex. The most characterized enzymes of this type are trypsin and chymotrypsin. Involvement of Ser proteases in apoptosis has been mostly studied by observing whether particular apoptotic events can be prevented by the specific inhibitors of these enzymes. In the early studies Gorczyca et al., (Gorczyca et al., Int J Oncol, 1992, 1:639-648) have shown that fragmentation of DNA in HL-60 cells treated with DNA topoisomerase inhibitors to induce apoptosis was prevented by irreversible inhibitors of Ser proteases such as diisopropylfluorophosphate (DFP), N-tosyl-L-phenylalanine chloromethyl ketone (TPCK) and N-tosyl-L-lysine chloromethyl ketone (TLCK), as well as by excess of the substrates N-tosyl-L-argininemethyl ester (TAME) and N-benzoyl-L-tyrosine ethyl ester (BTEE).

Concurrently, Bruno et al., (Bruno et al., Leukemia, 1992, 6:1113-1120; Bruno et al., Oncol. Res., 1992, 4:29-35) observed that the same inhibitors and substrates inhibited nuclear fragmentation as well as fragmentation of DNA in other cell types, including thymocytes treated with the corticosteroid prednisolone. It was also observed that these inhibitors prevented destabilization of double-stranded DNA (Hara et al., Exp Cell Res, 1996, 223:372-384), which during apoptosis becomes sensitive to denaturing agents and can be detected as single-stranded DNA (Hotz et al., Exp. Cell Res., 1992, 201:184-191). These initial observations were confirmed in many subsequent studies and in other cell systems (Hughes et al., Cell Death Differ., 1998, 5:1017-1027; Kim et al., Int. J. Oncol., 2001, 18:1227-1232; Ghibelli et al., FEBS Lett., 1995, 377:9-14; Lotem et al., Proc Natl Acad Sci USA, 1996, 93:12507-12; Mansat et al., FASEB J, 1997, 11:695-702; Gong et al., Cell Growth Differ, 1999, 10:491-502; Komatsu et al., J. Biochem (Tokyo), 1998, 124:1038-44; Yoshida et al., Leukemia, 1996, 10:821-4; Weaver et al., Biochem Cell Biol, 71:488-500; Park et al., Cytokine, 2001, 15:166-70). It should be noted, however, that while serine protease inhibitors prevent nuclear and DNA fragmentation triggered by different inducers, they themselves, especially after prolonged cell exposure, induce cell death that resembles apoptosis (Hara et al., Exp Cell Res, 1996, 223:372-384; Lu et al., Arch Biochem Biophys, 1996, 334:175-81).

The best recognized Ser proteases are granzymes A and B which are abundant in granules of cytotoxic T lymphocytes (CTL) and natural killer (NK) cells (Zapata et al., J. Biol. Chem., 1998, 273:6916-6920; Wright et al., Biochem. Biophys. Res. Commun., 1998, 245:797-803; Shi et al., J Exp Med, 1992, 176:1521-9; Kam et al., Biochim Biophys Acta, 2000, 1477:307-23; Jans et al., J Cell Sci, 1998, 111:2645-54; Estabanez-Perpina et al., J Biol Chem, 2000, 321:1203-1214). Granzyme B can cleave procaspase-3, −6, −7, −8, −9, and −10, and most likely, it activates endogenous caspases of the lymphocyte-target cells, thereby inducing their apoptosis (Zapata et al., J. Biol. Chem., 1998, 273:6916-6920). Granzyme A appears not to be associated with activation of caspases and it cleaves proteins independently of the latter (Shi et al., J Exp Med, 1992, 176:1521-9; Kam et al., Biochim Biophys Acta, 2000, 1477:307-23). Since granzymes A and B were studied predominantly in CTL or NK cells, it is unknown whether they play any role in apoptosis of other cell types.

Another apoptotic Ser protease is the 24-kD enzyme (AP24) shown to have the capacity to activate internucleosomal DNA fragmentation (Wright et al., J Exp Med, 1997, 186:1107-17; Wright et al., Cancer Res, 1998, 58:5570-6). Other Ser proteases that may function during apoptosis are the nuclear matrix-associated histone H1 specific enzyme induced by DNA damage (Kutsyi et al., Radiat Res, 1994, 140:224-229), the protease activated by Ca²⁺ (Zhivotovsky et al., Biochem Biophys Res Commun, 1997, 233:96-101) and myeoloblastin (Bories et al., Cell, 1989, 59:959-968). Most recently a new Ser protease, HtrA2/Omni, that is released from the mitochondria and interacts with the caspase inhibitor XIAP in a similar way as Smac/Diablo promoting cell death, have been identified (Suzuki et al., Molecular Cell, 2001, 8:613-621; Verhagen et al., J Biol Chem, 2001, 277:445-454; Martins et al., J Biol Chem, 2001, 277:439-444. It is unknown whether the Ser cathepsins A and G are involved in apoptosis although the cysteine cathepsin B and aspartate cathepsin D are present in lysosomes and endosomes and they may participate in heterophagic degradation of apoptotic bodies (Johnson et al., Leukemia, 2000, 14:1695-1703, Leist et al., Nature Rev Mol Cell Biol, 2001, 2:589-598).

Ser proteases also play an important role as markers of tumor malignancy. For example, several Ser proteases have been identified in prostate cells and their enzymatic activity has been shown to have a positive correlation with the development of prostate cancer as well as the degree of tumor malignancy (Yousef et al., J Biol Chem 2001, 276:53-61, Chen et al., J Biol Chem 2001, 276:21434-42, Takayama et al., Biochemistry, 2001, 40:1679-87, Magee et al., Cancer Res., 2001, 61:5692-6). Ser protease activity is also a diagnostic and prognostic marker in other tumors, such as breast carcinoma (Ulutin & Pak, Radiat Med 2000, 18:273-6,Yousef et al., Genomics, 2000, 69:331-41), and carcinomas of the head and neck (Lang et al., Br. J Cancer 2001, 84:237-43).

Activities of Ser proteases are also altered in a variety of other diseases. As mentioned, the Ser protease, granzyme B, is the key enzyme that is activated in a variety of cell-mediated immunological reactions. These cell-mediated responses include rejection of transplanted tissue (organs) and infections (Zapata et al., J. Biol. Chem., 1998, 273:6916-6920; Wright et al., Biochem. Biophys. Res. Commun., 1998, 245:797-803; Shi et al., J Exp Med, 1992, 176:1521-9; Kam et al., Biochim Biophys Acta, 2000, 1477:307-23; Jans et al., J Cell Sci, 1998, 111:2645-54; Estabanez-Perpina et al., J Biol Chem, 2000, 321:1203-1214).

The use of fluorochrome-labeled inhibitors of caspases (FLICA), to detect activation of these enzymes in living cells, has been reported (Bedner et al., Exp Cell Res., 2000, 259:308-313; Smolewski et al., Cytometry, 2001, 44:73-82; Darzynkiewicz et al., Methods Mol Biol (in press); Smolewski et al., Int J Oncol, 2001, 19:657-663). The FLICA are affinity labeling ligands that consist of carboxyfluorescein-tagged or sulforhodamine B-tagged peptide fluoromethyl ketones. They penetrate through the plasma membrane, covalently binding to active centers of caspases and at least during short-term incubations, remain relatively nontoxic to the cell. The amino acid sequence of the peptide residues that make up these reagents renders some binding selectivity toward the active center of the particular caspase. A good correlation was observed between activation of caspases detected by this assay and other markers of apoptosis (Bedner et al., Exp Cell Res., 2000, 259:308-313).

There is currently a need for novel assay methods that are useful for determining the apoptotic state of a cell. Such methods would be useful for detecting the presence of an apoptosis related disease in a subject. Such methods would also be useful for evaluating whether a therapeutic agent alters the apoptotic state of a cell.

### SUMMARY OF THE INVENTION

Applicant has discovered that the apoptotic state of a cell can be evaluated by measuring the level of caspase activity and the level of one or more active serine proteases in the cell. Due to the combined roles of caspases and serine proteases in apoptosis, the evaluation of the activity of both types of enzymes provides a better measure of the apoptotic state of a cell than the measurement of the activity of either type of enzyme alone.

Thus, the invention provides a method for determining the apoptotic state of one or more viable whole cells, comprising: 1) contacting the cells with a caspase affinity labeling agent and with a serine protease affinity labeling agent; and 2) detecting the presence of each affinity labeling agent in the cells; wherein the presence and relative abundance of the caspase affinity labeling agent and the presence and relative abundance of the serine protease affinity labeling agent correlate with the apoptotic state of the cells.

The invention also provides:
an assay reagent comprising a caspase affinity labeling agent and a serine protease affinity labeling agent; and a suitable carrier;
a method for detecting and/or predicting rejection of tissue or organ transplant where the presence or level (content) of the group L in the patient lymphocytes (“natural killer”; NK cells) or in cells of the transplanted organ (tissue) differs prior to- or at the time-of rejection from non-stimulated or pre-transplant tissue, by: 1) contacting the respective NK (or organ tissue) cells with the compound of invention; and 2) detecting the presence or relative abundance of the group L is predictive of the tissue rejection response or NK cell activation;
a method for diagnosis and prognosis assessment of other cell-mediated immunological reactions where the presence or relative abundance of the different group L detector molecules is characteristic of a particular type of cell mediated immunological reaction by; 1) contacting the cells with at least one compound of the invention, and 2) detecting the presence or relative abundance of the group L in the cells wherein the presence or relative abundance of L correlates with the detection and severity of the disease.

Preferably, the methods of the invention utilize a combination of reporter groups as exemplified in the following Table.

| | Red Serpase | Green Serpase | Cold Serpase |
|---|---|---|---|
| Red Caspase | + | +++ | ++ |
| Green Caspase | +++ | + | ++ |
| Cold Caspase | ++ | ++ | — |

Where
+ = claim;
++ = preferred;
+++ = most preferred.

Definitions:

Red caspase=e.g. sulforhodamine-VAD-FMK

Green caspase=e.g. fluorescein-VAD-FMK

Cold caspase=e.g. Z-VAD-FMK

Red serpase=e.g. sulforhodamine-FCK

Green serpase=e.g. fluorescein-FCK

Cold serpase=e.g. TPCK or TLCK

The invention also provides novel compounds of formula (I) and formula (II) disclosed herein, as well as salts thereof.

The invention provides methods which are useful for screening compounds, including libraries of chemical compounds, to identify therapeutic agents that modulate serine protease activity. The methods of the invention can be used to identify agents which induce, or reduce or inhibit apoptosis, as well as to identify therapeutic agents that are useful to treat diseases that are associated with serine protease activity. Techniques for screening chemical libraries are known in the art, and can be adapted for use in the methods described herein.

### BRIEF DESCRIPTION OF THE FIGURES

FIGS. 1A-1D. Illustrate the changes in the ability of HL-60 cells to bind 5(6)-Carboxyfluoresceinyl-L-valylalanylaspartylflyoromethyl ketone (FAM-VAD-FMK) and PI during apoptosis.

FIG. 2A-2H. Illustrate apoptosis-induced changes in the ability of HL-60 cells to bind 5(6)-Carboxyfluoresceinyl-L-phenylalanylchloromethyl ketone (FFCK) or 5(6)-carboxyfluoresceinyl-L-leucylchloromethyl ketone (FLCK).

FIGS. 3A-3B. Show the correlation between cell labeling with FAM-VAD-FMK and FFCK or FLCK.

FIGS. 4A-4C. Illustrate dual labeling of CPT-treated HL-60 cells with FFCK and Sulforhodaminyl-L-valylalanylaspartylflyoromethyl ketone (SR-VAD-FMK)

FIGS. 5A-5C. Illustrate dual labeling of CPT-treated HL-60 cells with FLCK and SR-VAD-FMK

### DETAILED DESCRIPTION

The following definitions are used, unless otherwise described. “Red” is a fluorescent dye such as a rhodamine, BODIPY, Cy dye, etc. which is excited by light >520 nm. “Green” is a fluorescent dye such as fluorescein, BODIPY FL or Cy-2 etc, which is excited around 488 nm. “Cold” refers to a group that does not fluoresce, is not colored, is not radioactive and which is not normally considered a hapten. Examples of “cold” groups include, but are not limited to tosyl and carbobenzyloxy (Z). Halo is fluoro, chloro, bromo, or iodo. Alkyl, denotes both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing 4 to 9 ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a dimethylene, trimethylene, or tetramethylene diradical thereto.

The term “serine protease affinity labeling agent” includes any agent capable of selectively binding, in a covalent manner, to one or more active serine proteases and facilitating their detection by analytical means. Accordingly, such an agent can include a florescent label, a radioactive label, or a hapten, or biotin as described herein. For example, one serine protease affinity labeling agent that can be used in the methods of the invention is a compound of formula I:
L-A-X—NH—CH(R′)C(═O)CH₂Cl (I)
wherein:
L is a detectable group;
A is a direct bond or a linker;
X is absent, an amino acid, or a peptide;
R′ is hydrogen, benzyl, 4-hydroxybenzyl, 3′-indolylmethyl, 2-methylpropyl, 1-methylpropyl, isopropyl, 4-aminobutyl, imidazolylmethyl or propylguanidino or (C₁-C₆)alkyl, wherein the alkyl is optionally substituted with one or more (1, 2, 3, or 4) substituents independently selected from the group consisting of guanidino, —C(═O)NRₐR_{b}, —C(═O)OR_{c}, halo, —NRₐR_{b}, aryl, heteroaryl, —OR_{c}, or —SR_{c};
each Rₐ and R_{b} is independently hydrogen, (C₁-C₆)alkyl, phenyl, benzyl, or phenethyl; or Rₐ and R_{b} together with the nitrogen to which they are attached form a pyrrolidino, morpholino, or thiomorpholino ring; and
each R_{c} is independently hydrogen, (C₁-C₆)alkyl, phenyl, benzyl, or phenethyl;
wherein any aryl or heteroaryl is optionally substituted with one or more (e.g. 1, 2, 3, or 4) substituents independently, selected from the group consisting of halo, nitro, cyano, hydroxy, mercapto, (C₁-C₆)alkyl, (C₁-C₆)alkoxy, trifluoromethyl, or trifluoromethoxy;
or a salt thereof.

For a compound of formula (I), L can preferably be a fluorescent label, a colored label, a radioactive label or hapten, or biotin; more preferably, L can be a fluorescent label (e.g. 5(6)-carboxyfluorescein, sulforhodamine B), or a colored label (e.g. 4-nitrophenyl or 2,4-dintrophenyl), or biotin. For a compound of formula (I), X can preferably be a peptide having about 2 to about 10 amino acids; more preferably, X can be a peptide having about 2 to about 5 amino acids.

Specifically, L is a fluorescent label, a colored label, a radioactive label, biotin or a hapten.

More specifically, L is a fluorescent label or biotin.

Preferably, L is 5(6)-carboxyfluorescein, or sulforhodamine B.

Specifically, X is a peptide containing from 2 to 10 amino acids. The amino acid composition of peptide X will define the enzyme selectivity of the affinity label. Enzymes will frequently target a 1 to 10 amino acid sequence identifying hydrophilic and hydrophobic residues within the sequence via complimentary amino acid sequences within the enzyme catalytic region. By selectively defining the composition of the peptide sequence, it has been shown that the target specificity of the enzyme substrate can be changed (Melo et al., Analytical Biochem, 2001, 293:71-77).

More specifically, X is a peptide having about 2 to 5 amino acids.

Preferably, X is an amino acid sequence consisting of: phenylalanine-proline (FP), phenylalanine-arginine (FR), isoleucine-alanine-methionine (IAM), alanine-alanine (AA), valine-proline (VP), glutamic acid-glycine (EG) or alanine-alanine-proline (AAP) dimers and trimers of glycine and alanine (GG, GGG, AA, and AAA), and dimers and trimers of a mixture of these amino acids (GA, GAA, GGA, GAG, AGG, AGA, AAG and AG).

Additionally, X can be an amino acid sequence that is a caspase target consisting of: VAD, YVAD, WEHD, VDVAD, DEHD, DEVD, WEHD, LEHD, VEID, LETD, AEVD, LELD and LEED (single letter abbreviations used are as follows; Ala (A), Arg (R), Asn (N), Asp (D), Cys (C), Glu (E), Gln (Q), Gly (G), His (H), Ile (I), Leu (L), Lys (K), Met (M), Phe (F), Pro (P), Ser (S), Thr (T), Trp (W), Tyr (Y), and Val (V)).

For a compound of formula (I), X can preferably be a natural amino acid (e.g. alanine, glutamic acid, valine); more preferably, X is absent.

For a compound of formula (I), R′ can preferably be benzyl, 2-methylpropyl, 1-methylpropyl, 4-aminobutyl, or propylguanidino (arginine).

In a more preferred embodiment of the present invention, R′ is hydrogen or (C₁-C₆)alkyl, wherein the alkyl is optionally substituted with one or more (1, 2, 3, or 4) substituents independently selected from the group consisting of guanidino, —C(═O)NRₐR_{b}, —C(═O)OR_{c}, halo, —NRₐR_{b}, aryl, heteroaryl, —OR_{c}, or —SR_{c}.

A preferred group of compounds of formula (I) are compounds wherein L is 5(6)-carboxyfluorescein, sulforhodamine B, or biotin; and R′ is benzyl, 2-methylpropyl, 1-methylpropyl, 4-aminobutyl, or propylguanidino (arginine).

A preferred compound of formula (I) is 5(6)-carboxyfluorescyl-L-phenylalanylchloromethyl ketone, 5(6)-carboxyfluorescyl-L-leucylchloromethyl ketone, or 5(6)-carboxyfluorescyl-L-lysylchloromethyl ketone; or a salt thereof. Other preferred compound groups of this invention would include fluorescein-5 or 6-isothiocyanate (FITC) and sulforhodamine labeled formulations of the same phenylalanyl, leucyl, or lysyl chloromethyl ketone compounds.

Preferred serine protease affinity labeling agents include the following compounds:

### 5(6)-Carboxyfluoresceinyl-L-phenylalanylchloromethyl ketone (FFCK)

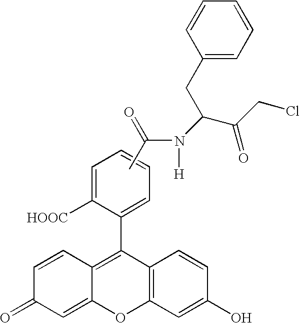

### 5(6)-Carboxyfluorescyl-L-leucylchloromethyl ketone (FLCK)

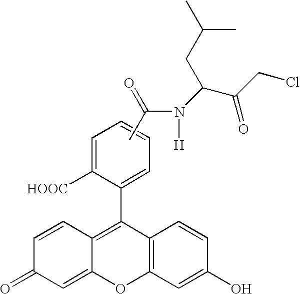

### 5(6)-Carboxyfluoresceinyl-L-lysylchloromethyl ketone

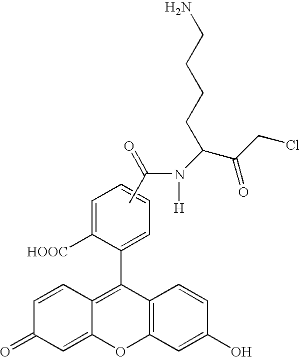

### 5(6)-Carboxyfluoresceinyl-L-arginylchloromethyl ketone

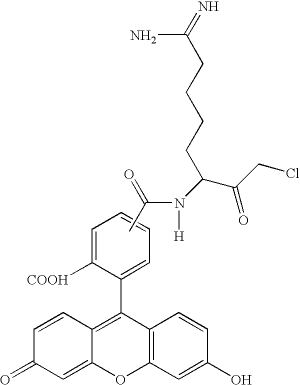

### Sulforhodarninyl-L-phenylalanylchloromethyl ketone

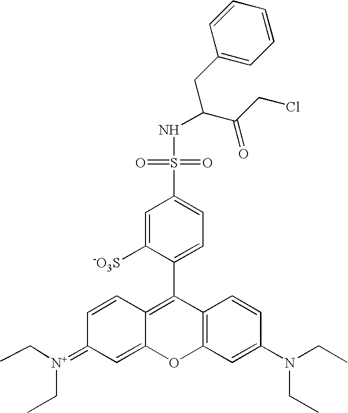

### Sulforhodaminyl-L-leucylchloromethyl ketone

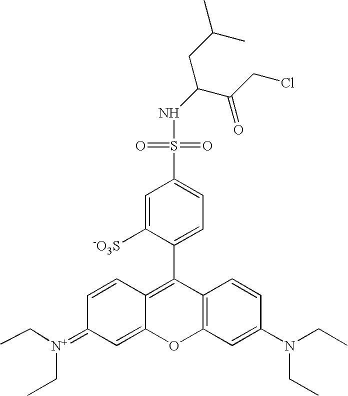

### Sulforhodaminyl-L-lysylchloromethyl ketone

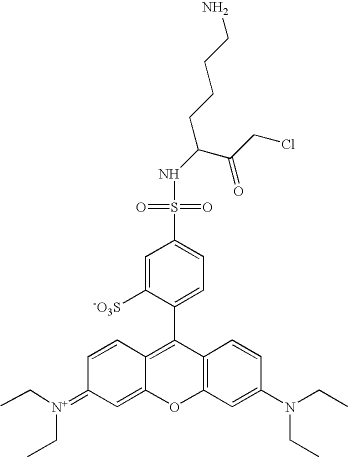

### Sulforhodaminyl-L-arginylchloromethyl ketone

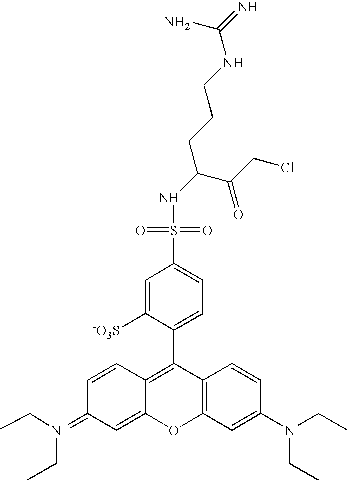

The term “caspase affinity labeling agent” includes any agent capable of selectively binding, in a covalent manner, to one or more active caspases and facilitating their detection by analytical means. The following table lists several caspase affinity labeling reagents and their corresponding caspase selectivity:

| Target Caspase | Product and Sequence | Other Sequences |
|---|---|---|
| Poly-Caspase | FAM-D-FMK | |
| Poly-Caspase | FAM-VAD-FMK | |
| Caspase-1 | FAM-YVAD-FMK | WEHD |
| Caspase-2 | FAM-VDVAD-FMK | DEHD |
| Caspase-3 and 7 | FAM-DEVD-FMK | |
| Caspases-4 and 5 | | (W/L)EHD |
| Caspase-6 | FAM-VEID-FMK | |
| Caspase-8 | FAM-LETD-FMK | |
| Caspase-9 | FAM-LEHD-FMK | |
| Caspase-10 | FAM-AEVD-FMK | LELD |
| Caspase-13 | FAM-LEED-FMK | |

Thornberry et al., Methods in Enzymology, 2000, 322: 100-125.

For example, such agents may include fluorescent labels (e.g. fluorescein derivatives, sulforhodamine derivatives, Cy dye derivatives, BODIPY derivatives, coumarin derivatives, or any fluorescent dye that can be attached to an amino group directly or by linkers), colored labels (e.g. 4-nitrophenyl or 2,4-dintrophenyl, or any colored label that can be attached to an amino group directly or by linkers), a radioactive label (e.g. tritium, carbon-14 phosphorus-32), or biotin, or a hapten (e.g. digoxigenin, and dinitrophenyl), or the like. Other labels such as biotin and the various high affinity binding type hapten groups (digoxigenin and dinitrophenyl) can be coupled to the affinity ligands to allow for the use of enzyme reporter group signal amplification. Commonly used enzymes include horseradish peroxidase (HRP), alkaline phosphatase (AP), β-galactosidase (BG), and urease (U). When coupled to avidin or IgG, for use in an avidin-biotin or hapten system respectively, the aforementioned enzyme molecules can convert colorless enzyme substrates to colored readout product. The most commonly used chromogenic substrates include tetramethylbenzidine (TMB) for use with HRP labels, and nitro blue tetrazolium/5-bromo-4-chloro-3-indolyl phosphate (NBT/BCIP) for use with AP labels. Commercial chemiluminescent substrates of these enzymes can also be used. Radioactive labels, such as tritium, carbon-14, and phosphorous-32 can be used as a direct label or can also be coupled to avidin or anti-hapten IgG for radioactive detection.

Other caspase affinity labeling agents would contain the same labels and 1 to 5 amino acid sequences but utilize an aldehyde modification of the aspartic terminal carboxyl group (HC═O), a chloromethyl ketone group (CH₂Cl), or an acyloxy reactive group ((C═O)O—Ar, where Ar is [2,6-(CF₃)₂]benzoate and various derivative of same (Krantz et al., Biochemistry, 1991, 30:4678-4687, and Thomberry et al., Biochemistry, 1994, 33:3934-3940).

For example, one caspase affinity labeling agent that can be used in the methods of the invention is the compound of formula II:
L₁-A₁-X₁—NH—CH(R₁′)C(═O)CH₂F (II)
wherein:
L₁ is a detectable group;
A₁ is a direct bond or a linker;
X₁ is absent, an amino acid, or a peptide; and
R₁′ is CH₂—COOH or CH₂CO₂R″, where R″ is methyl, ethyl, benzyl or t-butyl.

For a compound of formula (II), L₁ can preferably be a fluorescent label, a colored label, a radioactive label, a hapten or biotin; more preferably, L can be a fluorescent label (e.g. 5(6)-carboxyfluorescein, sulforhodamine B), or biotin. For a compound of formula (II), X₁ can preferably be a peptide having about 2 to 10 amino acids; more preferably, X₁ can be a peptide having about 2 to 4 amino acids (e.g. VA, YVA, DEV, LEE, LEH, VDVA, or AEV). For a compound of formula (II), X₁ can preferably be a natural amino acid (e.g. A, V, or E). The letter symbol V=valine, A=alanine, D=glutamic acid, L=leucine, and Y=tyrosine. For a compound of formula (II), R₁′ should be a methylene carboxy (ethanoic) side-chain (CH₂—COOH) as the caspases typically have a requirement for aspartate in the P₁ position of the peptide substrate. In a preferred configuration, the carboxyl groups of all aspartic and glutamic amino acid residues should exist as methyl esters of the carboxyl containing side-chains of —CH₂CO₂R, or CH₂CH₂CO₂R, where R is CH₃, other groups could include C₂H₅, C₄H₉, or CH₂C₆H₅ molecules.

A preferred compound of formula (II) is 5(6)-carboxylfluoresceinyl-L-valylalanylaspartylfluoromethyl ketone (FAM-VAD-FMK) or sulforhodaminyl-L-valylalanylaspartylfluoromethyl ketone (SR-VAD-FMK); or an ester thereof, or a salt thereof.

There are two main classes of α-amino acids: “natural” and “unnatural” α-amino acids. Additionally there are a wide variety of β-amino acids, homologues of amino acids and molecules that mimic amino acids, such as isosteres.

“Natural amino acids” refers to the naturally occurring α-amino acid molecules typically found in proteins. These are: glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine, and lysine.

“Natural amino acids” also exist in nature, which are not typically incorporated into naturally occurring proteins. Examples of these amino acids are: ornithine, γ-carboxyglutamic acid, hydroxylysine, citrulline, kynurenine, 5-hydroxytryptophan, norleucine, norvaline, hydroxyproline, phenylglycine, sarcosine, γ-aminobutyric acid and many others.

“Unnatural amino acids” are defined as those amino acids that are not found in nature and may be obtained by synthetic means well known to those schooled in amino acid and peptide synthesis. Examples of this class, which numbers in the many thousands of known molecules include: (t-butyl)glycine, hexafluoro-valine, hexafluoroleucine, trifluoroalanine, β-thienylalanine isomers, β-pyridylalanine isomers, ring substituted aromatic amino acids, at the ortho, meta, or para position of the phenyl moiety with one or more of standard groups of organic chemistry such as: fluoro-, chloro-, bromo-, iodo-, hydroxy-, methoxy-, amino-, nitro-, alkyl-, alkenyl-, alkynyl-, thio-, aryl-, heteroaryl- and the like.

It will be appreciated that amino acids and peptides can exist in L- or D-forms (enantiomers) and that certain amino acids with more than one chiral center, such as threonine, may exist in diastereomeric form. Further, when linked together in peptide chains, a mixture of L- and D-amino acids may be chosen to confer desired properties known in the art. Therefore, enantiomers, diastereomers and mixtures of these types are included in the claims.

Further, unnatural amino acids may exhibit other types of isomerism, such as positional and geometrical isomerism. These types of isomerism, coupled with or independent of optical isomerism, are also included in these claims.

In a specific preferred embodiment, the term “amino acid,” comprises the residues of the natural amino acids (e.g. Ala (A), Arg (R), Asn (N), Asp (D), Cys (C), Glu (E), Gln (Q), Gly (G), His (H), Hyl, Hyp, ile (I), Leu (L), Lys (K), Met (M), Phe (F), Pro (P), Ser (S), Thr (T), Trp (W), Tyr (Y), and Val (V)) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, -methyl-alanine, para-benzoylphenylalanine, phenylglycine, propargylglycine, sarcosine, and tert-butylglycine). When X is an amino acid in a compound of formula I, the amino terminus is on the left and the carboxy terminus is on the right.

The term “peptide” describes a sequence of 2 to 20 amino acids (e.g. as defined hereinabove) or peptidyl residues. Preferably a peptide comprises 2 to 10, or 2 to 5 amino acids. When X is a peptide in a compound of formula I, the amino terminus is on the left and the carboxy terminus is on the right.

It will be appreciated that the methods of this invention can be used with all cell types that contain or express serine proteases. The cells may come from plant, bacteria or animal origins and may be from tissue samples, fluid samples or immortalized cell lines. Cells originating from animals include cells from; Protozoa, Mastigophora or Flagellata, Sarcodina, Sporozoa, Cnidospora and Ciliata; Porifera; Coelenterata; Platyhelminthes; Pseudocoelomates, Rotifera, Gastrotricha and Nematoda; Molluska; Annelida; Arthropoda; Bryozoa; Eichinodermata; Chordata; Hemichordata; Vertabrates, Fishes, Amphibians, Reptiles, Birds and Mammals. More specific, Mammalian cells include but are not limited to cells such as lypmhocytes, neutrophiles, mast cells, neutrophiles, basophilic leukocytes, eosinophilic leukocytes, erythrocytes, monocytes, osteoblasts, osteoclasts, neurons, astrocytes, oligodendricites, hepatocytes, squamous cells, macrophages, fibroblasts, endothelial cells, chondrocytes, granulocytes, karyocytes, spermatocytes, spermatozoa, and cells of Sertoli. Imortalized cell lines include but are not limited to HL-60, MCF-7, Jurkat, U937, Hela, and THP-1.

The term “detectable group” includes any group that can be detected by analytical means. For example, suitable groups may be detectable by fluorescence spectroscopy, fluorescence microscopy, confocal fluorescence microscopy, fluorescence image analysis, flow cytometry, laser scanning cytometry, or plate multi-well fluorescence reader. Thus, suitable groups include florescent labels (e.g. fluorescein, rhodamines, Cy dyes, Bodipys, sulforhodamine 101, phycobiliproteins, etc. Other labels such as biotin and the various high affinity binding type hapten groups (digoxigenin and dinitrophenyl) can be coupled to the affinity ligands to allow for the use of enzyme reporter group signal amplification. Commonly used enzymes include horseradish peroxidase (HRP), alkaline phosphatase (AP), β-galctosidase (BG), and urease (U). When coupled to avidin or IgG, for use in an avidin-biotin or hapten system respectively, the aforementioned enzyme molecules can convert colorless enzyme substrates to colored readout product. The most commonly used chromogenic substrates include tetramethylbenzidine (TMB) for use with HRP labels, and nitro blue tetrazolium 5-bromo-4-chloro-3-indolyl phosphate (NBT/BCIP) for use with AP labels. Commercial chemiluminescent substrates of these enzymes can also be used. Radioactive labels, such as tritium, carbon-14, and phosphate-32 can be used as a direct label or can also be coupled to avidin or anti-hapten IgG for radioactive detection.

The nature of the “linker” is not critical provided the final compound of formula I has suitable properties (e.g. suitable solubility, cell toxicity, cell permeability, and ability to selectively react with the targeted serine protease group) for its intended application. The linker, denoted by the letter “A”, in the case where “A” can simply be a covalent bond, the detectable group (L) is attached directly to the N-terminal amino group of the peptide or amino acid (e.g., amide linkage L-(C═O)—NH—R). “A” can also be any member of the class of linkers well known to those experienced in this field. Linkers are typically 4-18 atoms long, consisting of carbon, nitrogen, oxygen or sulfur atoms. Specific examples of linkers include ε-aminocaproic acid (6 atoms), di-ε-aminocaproic acid (12 atoms), oligomers of ethylene glycol (—O—(CH₂CH₂O)ₙCH₂CH₂—, where n=0-5); or di- and triamines separated by 2 to 6 methylene groups, for example: —HN(CH₂)ₙ—NH(CH₂)ₘ—NH(CH₂)ₒ— where n, m and o are integers from 0 to 6. Typical linkers include ester (—OC(═O)—), thioester (SC(═O)—), thionoester (—OC(═S)—), carbonyl (—C(═O)—), and amide (—NHC(═O)—) groups, as well as divalent phenyl groups, and a 1 to 10 membered carbon chain, which chain can optionally comprise one or more double or triple bonds, and which chain can also optionally comprise one or more oxy (—O) or thioxy (—S—) groups between carbon atoms of the chain. A preferred linker is a simple amide linkage ((—NHC(═O)—) or —C(═O)NH—) facilitated by an activated carboxyl-N-hydroxysuccinimide leaving group coupling system.

The assay reagents of the invention can also comprise one or more suitable carriers. Suitable carriers include polar, aprotic solvents (e.g. acetonitrile, DMSO or DMF) or protic solvents (e.g. water, methanol, ethanol, etc.).

The term “active serine protease” is defined as an active enzyme representative of a family of proteases which utilize serine as the electron exchange group. An “active serine protease” is an enzyme which is in its catalytically active form. Some examples of this type of enzyme includes the known apoptosis-associated Ser proteases such as A24, granzymes A and B, Cathepsins A and G, HtrA2/Omni protease, as well as numerous yet unrecognized proteases that become activated during apoptosis. This term also includes other Ser proteases such as those associated with prostate tissue or cancer (prostate specific antigen (PSA), hepsin, prostasin, etc.) and with other tissues and organs.

The term “agent that promotes cell death” is defined as those agents whose function is to disrupt the normal stasis condition of the cell beyond which the cell can accommodate and recover. This pushes the cell to undergo apoptosis, and eventual cell death. Anti-cancer treatment agents fall into this classification. They are used in an attempt to reduce the rate of cancer cell proliferation and at the same time, induce the target cancer conversion to apoptosis. All of these anti-cancer therapeutic agents are designed to induce cellular stress by targeting key cellular structures such as the DNA, lipid component of the cell membranes, and key cellular proteins responsible for maintaining the metabolic equilibrium (stasis). When the damage exceeds the ability of the cells to make adjustments and repairs, then apoptosis often ensues. The table below provides several examples of some key target mechanisms a long with their respective therapeutic agents:

| Mechanism | Therapeutic Agents |
|---|---|
| DNA Damaging Reagents | Cyclophosphamide, Cisplatin, Doxorubicin, |
| | Ionizing Radiation |
| Anti-metabolites | Methotrexate, 5-Flurouracil, 5-Azacytidine |
| Mitotic Inhibitors | Vincristine |
| Nucleotide Analogs | 6-Mercaptopurine |
| Topoisomerase Inhibitors | Etoposide, Camptothecins |

Herr et al., Blood, 2001, 98: 2603-2614.

The term “topoisomerase inhibitor” is defined as those reagents, which bind to either Type I or Type II topoisomerases, causing errors in DNA replication leading to induction of apoptosis (Juo, Concise Dictionary of Biomedicine and Molecular Biology, 1996). Camptothecin is an example of a topoisomerase I inhibitor. This reagent binds to the DNA-topoisomerase I complex, interfering with the DNA unfolding process. Etoposide also interferes with DNA synthesis by inducing double and single strand breakage via inhibition of topoisomerase II (Hertzberg et al., J. Biol Chem, 1990, 265:19287).

The term “agent that protects the cell from cell death” includes all the treatments whose strategy is to prevent cell apoptosis. They include scavengers of the reactive oxygen species (radicals) such as acetylcysteine, etc., agents and treatments that down-regulate the pro-apoptotic members of Bcl-2 family of proteins or up-regulate the anti-apoptotic members of the Bcl-2 family.

The term “apoptotic state of a cell” means the current status of the cell, whether it continues to be functioning normally, or entering into the various characteristic stages of the apoptotic process. Cells usually progress through the process of apoptosis, generally showing one or more features (morphological, biochemical or molecular) characteristic of apoptosis.

The term “induces apoptosis” means the treatment that commits and/or preconditions the cell to enter the apoptotic process.

The term “reduces or inhibits apoptosis” means the treatment causes a reduction in the eventuality or probability of the cell entering the process of apoptotic or prolongs or halts the process itself. This is important when considering treatments for neurodegenerative disease such as Alzheimers disease (AD). Alzheimers disease is a neurodegenerative disease characterized by a progressive memory loss and increasing levels of dementia. One of the key pathological features of the disease is the expression of a high frequency of extracellular plaques which are formed from the deposition of amyloid β (Aβ) peptides that are derived from (Aβ) protein precursor (AβPP). Caspase-6 is capable of cleaving AβPP and the presenilins. It is also localized to pathological lesions associated with AD. Upstream caspases such as caspases-8 and 9 are also elevated in the AD neurons. Given the association of caspases with the active form of this disease, treatment strategies have evolved around the use of caspase inhibitors that transverse the cell membrane. The earliest therapeutic inhibitor agents consisted of benzyloxycarboxyl-L-valylalanylaspartylfluoromethyl ketone (z-VAD-FMK) and benzyloxycarboxyl-L-tyrosinylvalylalanylaspartylfluoromethyl ketone (z-YVAD-FMK). These inhibitors form covalent linkages with a SH-cysteine within the caspase reactive centers, thus inactivating the caspase enzyme activity. A number of pharmaceutical companies are using this attack strategy in their development of peptoid inhibitors and non-peptide inhibitors such as the Isatin Sulfonamides. Other and as yet undefined therapeutic strategies would include up-regulation of the anti-apoptotic members of the Bcl-2 family (e.g. Bcl-xL and Bcl-W) and conversely down regulate the pro-apoptotic Bcl-2 membership proteins such as Bax, Bak, Bok, or Bid, Bad, Bih as example. Nicholson et al., Nature, 2000, 407:810-815; Raina et al., Acta Neuropathol, 2001, 101:305-310; and Lee et al., J. Med Chem, 2001, 44:2015-2026.

The term “necrosis” means the alternative, disorderly mode of cell death. Cells undergoing necrosis usually swell up and burst, releasing the cytoplasmic contents into the surrounding environment. Necrotic cell death does not require the energy derived from ATP.

The term “relative abundance” can be defined as; 1) the amount of fluorescent label observed in stimulated cells or tissue compared to the non-stimulated cells or tissue, 2) the ratio of one fluorescently labeled affinity ligand to the other fluorescently labeled affinity ligand in stimulated versus non-stimulated cells or tissue, 3) the amount of fluorescent label observed in disease state cells or tissue compared to normal/healthy cells or tissue, and 4) the ratio of one fluorescently labeled affinity ligand to the other fluorescently labeled affinity ligand in disease state cells or tissue versus normal/healthy cells or tissue.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentyloxy, 3-pentyloxy, or hexyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The assay reagents of the invention can also comprise one or more suitable carriers. Suitable carriers include DMSO, DMF, or other organic solvents which, when diluted out in aqueous buffer media, present minimal toxicity to the cell system being analyzed.

In cases where the affinity labels are sufficiently basic or acidic to form stable acid or base salts, use of the compounds as salts may be appropriate. Examples of such salts are organic acid addition salts formed with acids which form an acceptable anion, for example, tosylate, methanesulfonate, acetate, citrate, malonate, tartarate, succinate, benzoate, ascorbate, αketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording an acceptable anion. Alkali metal (for example, sodium, potassium or lithium) or alkaline earth metal (for example calcium) salts of carboxylic acids can also be made.

The invention will now be illustrated by the following non-limiting Examples.

### EXAMPLE 1

General Protocol for Use of Novel Affinity Labels

Preparation of reagents: Fluorescent inhibitors of serine proteases (FLISP) reagents, 5(6)-Carboxyfluoresceinyl-L-phenylalanylchloromethyl ketone (FFCK) and 5(6)-carboxyfluoresceinyl-L-leucylchloromethyl ketone (FLCK) were dissolved initially in dimethyl sulfoxide (DMSO; Sigma) to yield a 10 mM concentration. Aliquots were made from this stock solution and stored frozen at −20° C. protected from light. This reagent stock was then diluted directly into the cell culture media to give a 1× working reagent concentration of 10 μM. Dilution of the reagent into aqueous (cell culture) media is done just prior to cell exposure to preserve the labile chloromethyl ketone reactivity of FLISP reagent.

Fluorescent inhibitors of caspases (FLICA) reagents, namely the fluorescein labeled VAD-FMK (FAM-VAD-FMK) and sulforhodamine labeled VAD-FMK (SR-VAD-FMK) (Immunochemistry Technologies; Bloomington, Minn.) were both designed to detect the presence of active caspases within apoptotic cells. These inhibitors were dissolved in DMSO to obtain a 150× concentrated stock solution. Aliquots of these solutions were stored at −20° C. in the dark. Prior to use, a 30× working solution of either FAM-VAD-FMK or SR-VAD-FMK was prepared by diluting the stock solution 1:5 in phosphate buffered saline (PBS) and mixing until the solution become clear. The 30× working solution was diluted 1:30 cell culture media to give a final 1× working reagent concentration of 10 μM.

Unlabeled (cold) N-tosyl-phenylalanylchloromethyl ketone (TPCK) and N-tosyl-lysylchloromethyl keytone (TLCK) were obtained from Sigma Chemical Co.; concentrated solutions at 10 mM were freshly prepared in DMSO. Further dilutions were made in tissue culture media.

The non-fluorescent poly-caspase inhibitor Z-VAD-FMK was obtained from Enzyme Systems Products. A 20 mM stock solution of Z-VAD-FMK was made in DMSO (Sigma) and the inhibitor was then diluted in culture media to obtain the final 50 μM concentration in the cultures.

Cells: Human promyelocytic leukemic HL-60 cells were obtained from American Type Culture Collection (ATCC; Rockville, Md.). They were cultured in 25 mL FALCON flasks (Becton Dickinson Co., Franklin Lakes, N.J.) using RPMI 1640 supplemented with 10% fetal calf serum, 100 units/mL penicillin, 100 mg/mL streptomycin and 2 mM L-glutamine (all from Gibco/BRL Life Technologies, Inc., Grand Island, N.Y.) in a humidified incubator set to maintain 37.5° C. and 5% CO₂ as previously described (Bedner et al., Exp Cell Res., 2000, 259:308-313; Smolewski et al., Cytometry, 2001, 44:73-82). At the onset of experiments, there were fewer than 5×10⁵ cells/ml in culture. To induce apoptosis the cells were treated with 0.15 μM DNA topoisomerase I inhibitor camptothecin (CPT; Sigma Chemical Co., St. Louis, Mo.) for 3 hours.

Cell staining and fluorescence measurement by LSC: The HL-60 cells from the untreated or CPT treated cultures were centrifuged (200 g, 5 min) and resuspended in PBS at approximately 10⁴ cells per 5 mL volume. Cells were then attached electrostatically to microscope slides as described before (Bedner et al., Exp Cell Res., 2000, 259:308-313; Smolewski et al., Cytometry, 2001, 44:73-82). The attachment was achieved by the incubation (15 min) of cells suspended in serum-free PBS in shallow (<1 mm depth; 1.5×1.5 cm) wells on horizontally placed microscope slides that were rinsed in 100% ethanol and air dried prior to use, at 100% humidity. The electrostatically attached cells remain viable, exclude such dyes as trypan blue and propidium iodide (PI), and have unchanged morphology for several hours (Bedner et al., Exp Cell Res., 2000, 259:308-313). After the cells became attached, PBS was removed from the wells and was replaced by 150 μL of the culture medium containing 10% FCS. FLISP staining solutions were prepared by diluting 5 μL of the 10 mM FFCK or FLCK stock solution into 5 mL of culture medium yielding a final FLISP concentration 10 μM. The medium from above the cells on the slide was then replaced with 150 μL of this staining solution. A polyethylene foil (2.5×2.5 cm) was positioned over the staining solution to prevent drying. The slides were subsequently incubated for 1 h at 37° C. in a closed box with wet tissue to additionally prevent drying. The FLISP staining solution was removed by immersing the slides for 2 min in PBS in Coplin jars, containing fresh PBS. The washing step was repeated once more with fresh PBS. If desired, a 100 μL aliquot of PBS solution containing 0.1 μg of propidium iodide (PI; Molecular Probes, Eugene, Oreg.) can be layered atop the cells and the specimen was covered with a glass coverslip (if PI is not used, layer 100 μL of PBS atop the cells). The slides were placed on the motorized stage of laser scanning microscope (LSC) for fluorescence measurement. Cell fluorescence was then measured using a 488 nm excitation laser line and recording integral and maximal pixel intensities of the green FFCK or FLCK. FLICA staining was measured under the same conditions as FLISP staining. Fluorescence can also be measured by flow cytometer and fluorescence microscopy.

### EXAMPLE 1A

### Correlation Between CPT Apoptosis Induction and Binding of FFCK, FLCK and Caspase Detector, FAM-VAD-FMK.

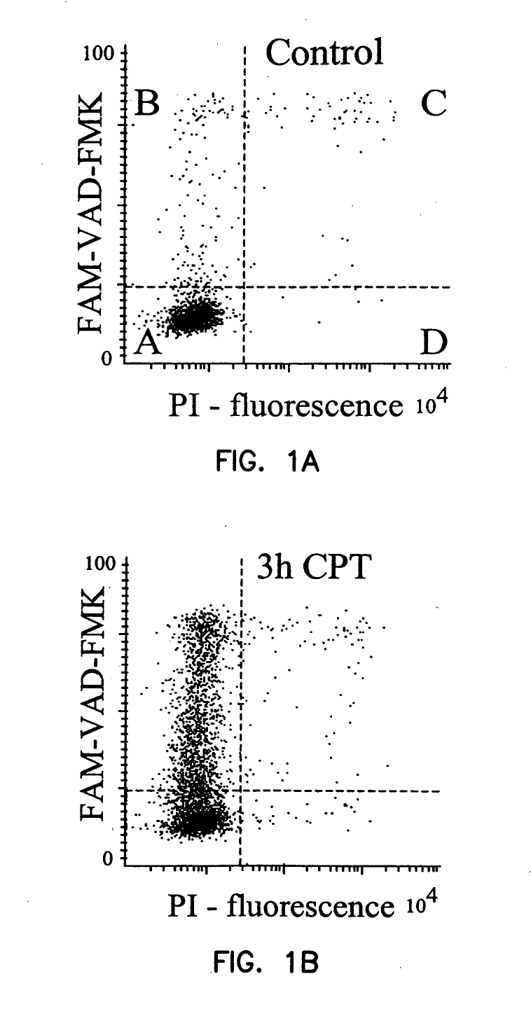

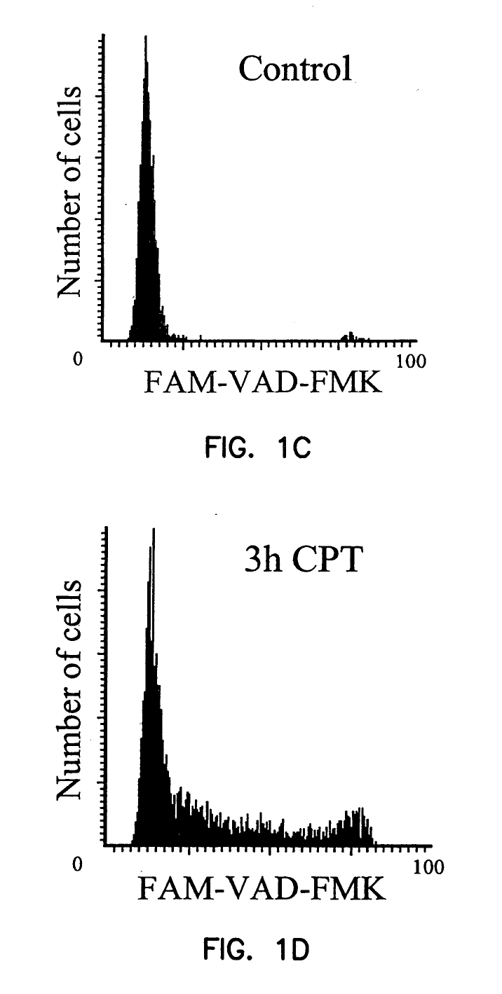

FIG. 1 illustrates changes in the capability of HL-60 cells treated with CPT to bind FAM-VAD-FMK and PI. Based on observable fluorochrome binding differences, four cell subpopulations were identified on the bivariate PI (red) vs FAM-VAD-FMK (green) fluorescence distributions (scatterplots) Table 1:

TABLE 1

| Quadrant | Fluorescence | Cell State |
|---|---|---|
| A | FLICA−/PI− | Non-Apoptotic Cells |
| B | FLICA+/PI− | Early Apoptotic Cells |
| C | FLICA+/PI+ | Late Apoptotic Cells |
| D | FLICA−/PI+ | Very Late Apoptotic or Necrotic Cells |

The FLICA−/PI− cells were most frequent (>95%) in the untreated, control cultures. The CPT treatment initially led to a marked increase in percentage of FLICA+/PI− (FIG. 1), which was later followed by the appearance of FLICA+/PI+ and then FLICA+/PI−, cells. Activation of caspases was an early event, followed later by the loss of plasma membrane ability to exclude PI.

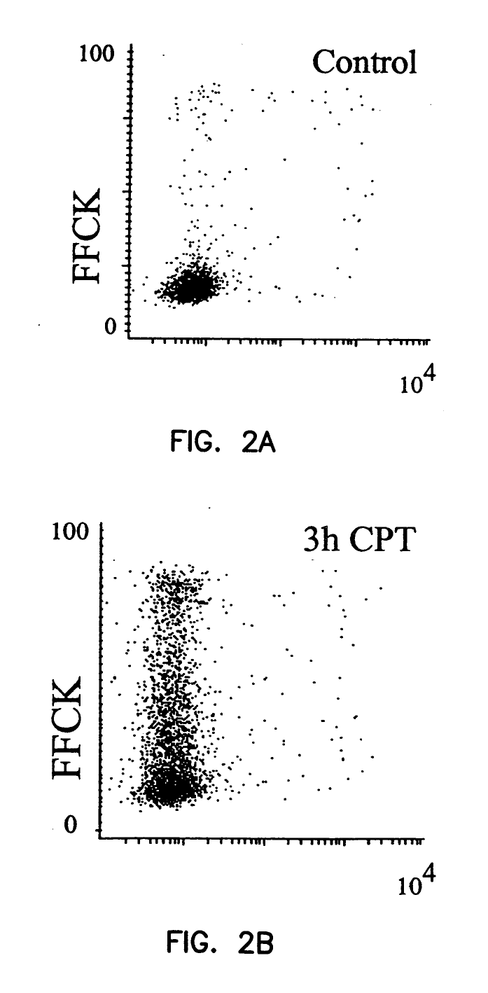

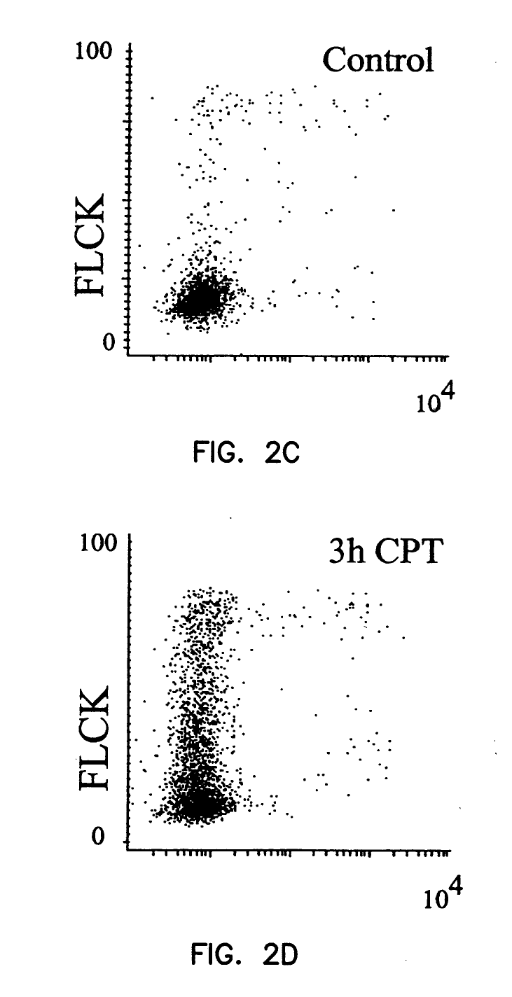

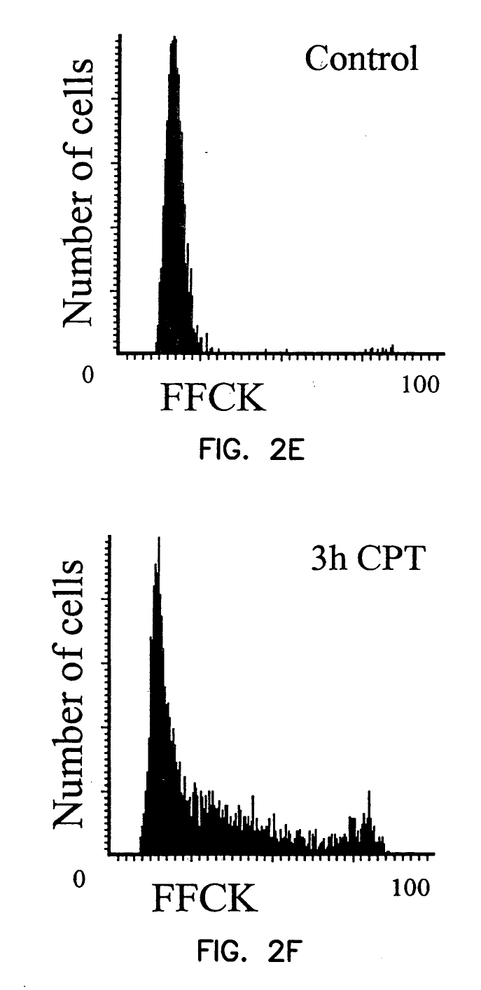

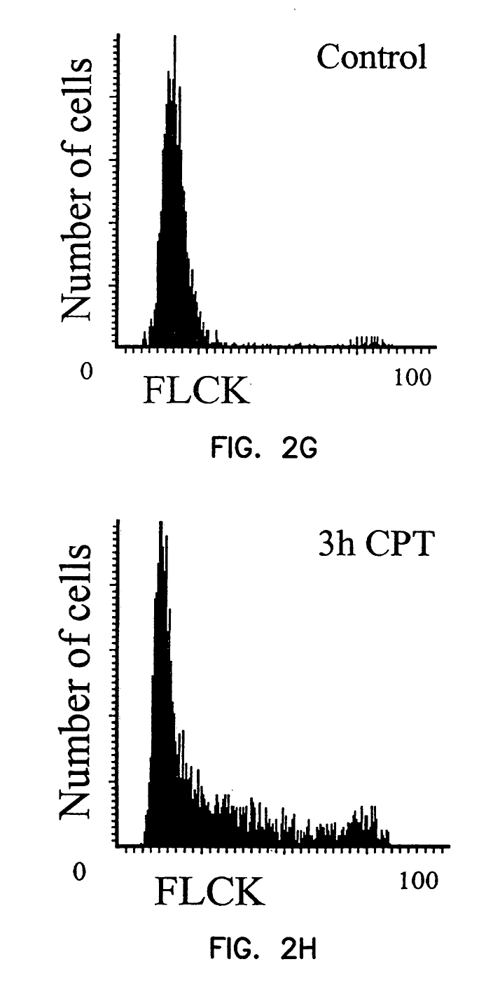

FIG. 2 shows the binding of FFCK or FLCK, each combined with PI, by the untreated (control) cells and by the cells treated for 3 h with CPT. It is apparent that treatment with CPT induced binding of both ligands. In analogy to cultures subjected to FAM-VAD-FMK and PI binding (FIG. 1) relatively few cells become labeled with PI in the cultures after 3 h CPT exposure and assay using FFCK or FLCK (FIG. 2).

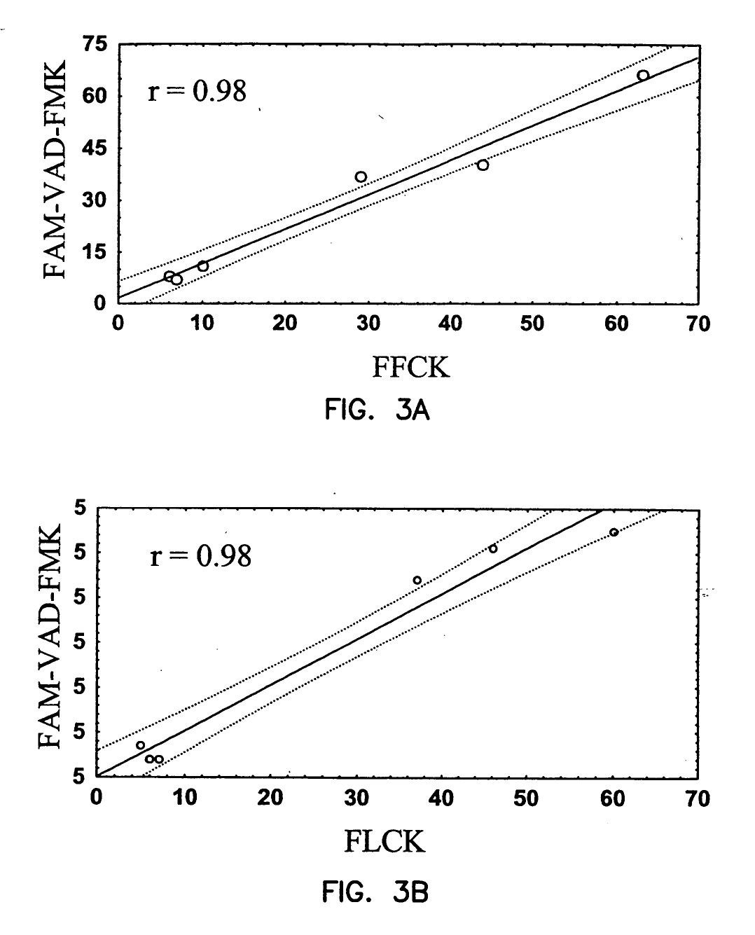

FIG. 3 represents the repeated analysis of the untreated and CPT-treated cultures with respect to the frequency of FAM-VAD-FMK vs FFCK or FLCK labeled cells that showed a high degree of correlation. Such a correlation suggests that activation of caspases detected by FAM-VAD-FMK binding occurred in the same cells that reacted with FFCK or FLCK. Also, the time-frame during which the cells remained reactive with each of these probes, appeared to be of similar length.

### EXAMPLE 1B

### Sequential Activation of Caspases and Serine Proteases During Apoptosis

Experiments were conducted to reveal whether activation of caspases and appearance of the FFCK or FLCK binding sites depend on each other. Towards this end the cells were treated with CPT in the presence or absence of the unlabeled poly-caspase inhibitor Z-VAD-FMK for 3 h and then assayed for activation of either FFCK or FLCK binding sites. And conversely, the cells induced to apoptosis by CPT were maintained in the presence or absence of either unlabeled TPCK or TLCK and activation of their caspases was subsequently assayed by FMK-VAD-FMK binding. The results of these experiments are shown in Table 2.

TABLE 2
Effect of the pretreatment of HL-60 cells during induction of apoptosis
with Z-VAD-FMK, TPCK or TLCK on the subsequent binding of
FAM-VAD-FMK, FFCK and FLCK.

| Pretreatment | FAM-VAD-FMK | FFCK | FLCK |
|---|---|---|---|
| Z-VAD-FMK | 95.0 | 83.0 ± 3.6 | 77.2 ± 3.2 |
| TPCK | 27.7 ± 5.8 | 94.2 | 38.9 ± 1.7 |
| TLCK | 0.5 ± 5.2 | 2.2 ± 2.3 | 1.74 ± 4.6 |

The data in Table 2 show percent decrease in frequency of the labeled cells pre-treated with the unlabeled protease inhibitors compared to the respective controls, namely to the cells treated with CPT in the absence of the unlabeled inhibitors. Between 3,000-10,000 cells were recorded per each measurement. Mean values SE) of three independent experiments are presented.

It is apparent that pretreatment with Z-VAD-FMK quite effectively prevented the appearance of either FFCK or FLCK binding sites, as the cell labeling with these ligands was reduced by 83.0 or 77.2%, respectively. Compared to Z-VAD-FMK, the protective effect of TPCK was less pronounced. Namely, the FAM-VAD-FMK- or FLCK-reactivity of cells pre-treated with TPCK was diminished only by 27.7 or 38.9%. However, unlabeled TPCK prevented the subsequent binding of its fluorescein-conjugated analog by as much as 94.2%. TLCK offered no protection at all for the subsequent binding of either FAM-VAD-FMK, FFCK or FLCK.

### Experiment 1C. Dual Labeleing With SR-VAD-FMK and FFCK or FLCK

The availability of the red fluorescing poly-caspase inhibitor SR-VAD-FMK and green fluorescing FLISP reagents, offered an opportunity to compare, within the same cells, labeling of activated caspases vis-à-vis the FFCK and FLCK binding sites. When examined by fluorescence microscopy or imaged by LSC, it was seen that fluorescence of induced cells treated simultaneously with SR-VAD-FMK and FFCK (or FLCK) was primarily restricted to the cells that showed morphological changes characteristic of apoptosis. These changes included overall cell shrinkage as well as shedding of apoptotic bodies (“budding” of the plasma membrane) into the surrounding media. Essentially all such cells were fluorochrome-labeled. In contrast, few cells (<10%) with unchanged morphology were labeled.

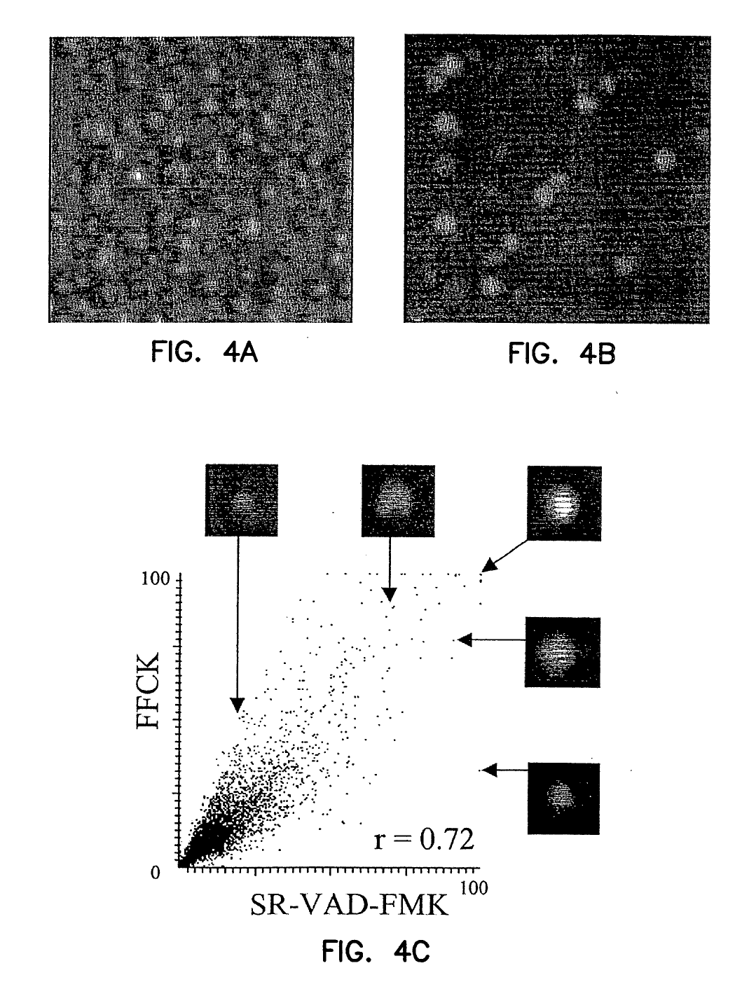

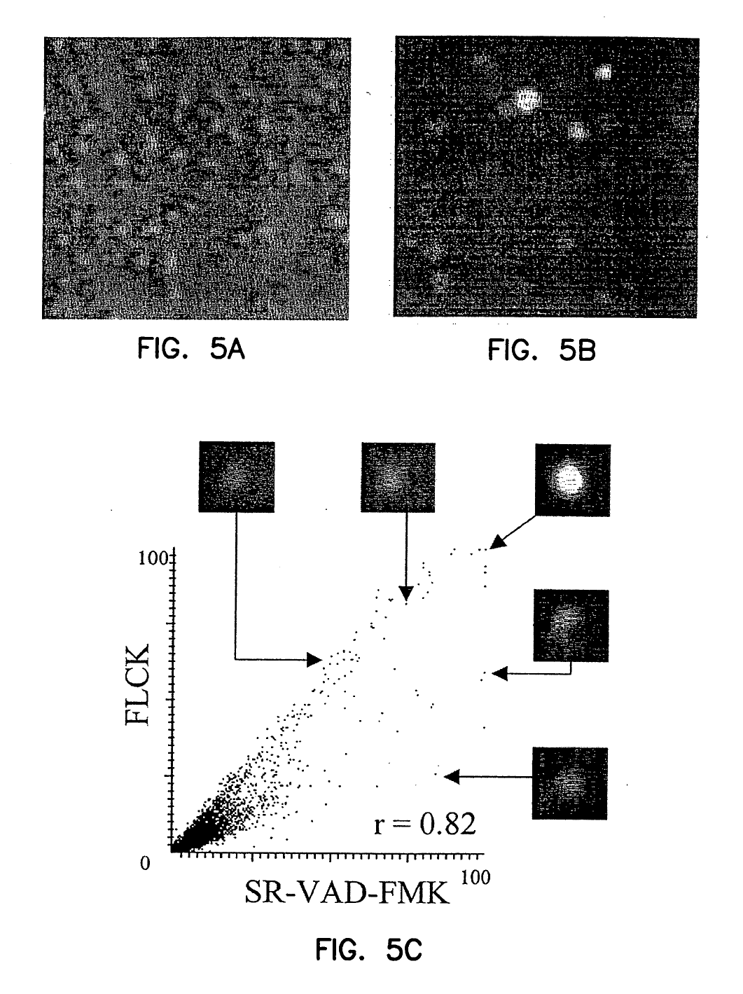

FIGS. 4 and 5 reveal an interesting pattern of significant variability in overall proportions of the sites reactive with SR-VAD-FMK vs FFCK or FLCK in individual cells, as well as in their intracellular localization. Some cells displayed prominent green- or red-fluorescence while others fluoresced in various hues of yellow. This heterogeneity was mirrored by a widely scattered distribution plotting of individual cells on the bivariate scatterplots representing intensity (integral values) of cellular red (SR-VAD-FMK) vs green (FFCK or FLCK) fluorescence. The green fluorescence of FFCK was strong and often localized in the cytoplasm in a single or two distinct and relatively large perinuclear foci. Also, nucleoli were frequently labeled with FFCK. Fluorescence of cells treated with FLCK was faint and more uniformly distributed. The red fluorescence of SR-VAD-FMK was uniformly dispersed.

It was shown before (Bedner et al., Exp Cell Res., 2000, 259:308-313) that frequency of cells reactive with FAM-VAD-FMK was strongly correlated with the fraction of apoptotic cells identified by the presence of DNA strand breaks (r=0.96). A strong correlation was seen between the percentage of cells labeled with FAM-VAD-FMK (or SR-VAD-FMK) and either with FFCK or FLCK (FIGS. 3-5). It is quite evident that the ability of cells to bind either FFCK or FLCK concurred with induction of the binding of the poly-caspase labeled inhibitor FAM-VAD-FMK (or SR-VAD-FMK) and both reactivities were markers of apoptosis.

Induction of apoptosis in HL-60 cells by CPT led to a rapid increase in binding of FFCK or FLCK concomitant with binding of FAM-VAD-FMK (or SR-VAD-FMK). The fraction of cells labeled with each of these ligands was similar, varying after 3 h of treatment with CPT, between 35-45% in repeated experiments, and generally approximating the percentage of the S-phase cells in these cultures. Most labeled cells showed signs typical of apoptosis.

The present invention provides novel fluorochrome-labeled affinity markers of the enzymatic centers of serine proteases (e.g. FFCK and FLCK). It was proposed that if serine proteases are activated during cellular processes their active sites may become accessible to these ligands. Indeed, it was found that during apoptosis the sites reactive with FFCK and FLCK become accessible and reacted with these inhibitors. Most likely, the binding is covalent because it withstands subsequent cell fixation, permeabilization and rinses.

The following evidence is consistent with the assumption that the observed binding was indeed specific to enzymatic centers of Ser proteases and thus signaled their intracellular activation:
(1) Analogs of FFCK and FLCK ligands (e.g. TPCK) exhibit a high affinity interaction with the active centers of the chymotrypsin-like enzymes, binding covalently via the alkylation of the imidazole ring of His-57 (Shaw et al., Biochem Biophys Res Commun., 1967, 27:391-7; Blow, D. M., Acc Chem Res, 1976, 9:145-152; Wilcox, P. E., Methods Enzymol, 1970,19:64-108). As such, they are widely used as specific inhibitors of these enzymes. Indeed, it was observed that TPCK (TFCK, using current amino acid symbols) prevented binding of FFCK (Table 2), indicating that both ligands compete for the same sites;
(2) Prior cell exposure to TLCK during induction of apoptosis did not prevent the subsequent binding of FAM-VAD-FMK. Pre-exposure to TPCK had only a modest suppressive effect on the FAM-VAD-FMK binding (Table 2). This evidence suggests that despite the similarity of the reactive moieties (halomethyl ketone) the binding sites of FAM-VAD-FMK and FLISP are different; FLISP reagents do not bind to caspases and serine proteases do not bind FLICA reagents; the binding sites are different because they are different enzymes;
(3) The intracellular localization of the enzymes detected by SR-VAD-FMK and FFCK or FLCK in many cells was distinctly different (FIGS. 4 and 5);
(4) Dual cell labeling with SR-VAD-FMK and FFCK or FLCK led to a mixed ratio of red to green fluorescence within individual cells. Some cells exhibited a red fluorescence, while others displayed a green fluorescence and still others fluoresced yellow (FIGS. 4 and 5). Were the same enzymatic sites reacting with SR-VAD-FMK and FFCK or FPCK, all cells would be uniformly stained, with equal mixtures of red and green fluorescence. Given the above, the binding sites that become accessible to FFCK and FLCK during apoptosis cannot be of the activated caspases. Furthermore, FFCK and FLCK do not have the requisite aspartic acid residue for optimal caspase binding and they are optimally designed for chymase binding, so these results fit with expectations. It is likely, therefore, that the observed binding of these ligands reflects the increased accessibility of the enzymatic centers of Ser proteases. As mentioned above, there is strong evidence that several Ser proteases undergo activation during apoptosis; among them AP24 (Wright et al., Biochem. Biophys. Res. Commun., 1998, 245:797-803; Wright et al., J Exp Med, 1997, 186:1107-17; Wright et al., Cancer Res, 1998, 58:5570-6)and HtrA2/Omni (Suzuki et al., Molecular Cell, 2001; 8:613-621; Verhagen et al., J Biol Chem, 2001, 277:445-454; Martins et al., Biochem Biophys Res Commun, 1998, 245:797-803) are the best characterized.

From the present data, FFCK and FLCK do not bind to the active centers of the same enzymes and therefore it is possible that detection of the activation of two different serpases of the chymotrypsin-like family (chymases) occurred. FFCK, having a Phe moiety, is expected to be a specific inhibitor of chymotrypsin (EC 3.4.21.1). FLCK, with a Leu moiety, should have preference to chymotrypsin C (EC 3.4.21.2) (Blow, D. M., Acc Chem Res, 1976,9:145-152; Wilcox, P. E., Methods Enzymol, 1970, 19:64-108).

Additional support for the notion that activation of two enzymes has been detected was provided by the observation that the pattern of cell labeling with FFCK and SR-VAD-FMK was different than that observed using FLCK and SR-VAD-FMK (FIGS. 4 and 5). Furthermore, while pretreatment with TPCK prevented the subsequent binding of FFCK by 94.2% it had lesser effect (38.9% suppression) on the binding of FLCK (Table 2). Also different, was the absolute intensity of cell fluorescence after labeling with either FFCK or FLCK. Namely, when measured under identical settings of the photomultiplier sensitivity, the FLCK labeled cells had approximately 60% greater fluorescence intensity compared to the cells labeled with FFCK. All this evidence supports the concept that the labeled inhibitors FFCK and FLCK did not compete for the same binding sites and thus, most likely, are bound to separate enzymes.

Activation of each of the sites, the one reactive with FFCK, and the other, with FLCK, appeared to depend on a prior caspase activation event. This transpired from results of the experiments showing that binding of these ligands was greatly diminished when the poly-caspase inhibitor Z-VAD-FMK was present in the media during CPT stimulation. In contrast, activation of caspases was unaffected by TLCK and only modestly suppressed by TPCK (Table 2). TLCK, having the charged amino acid Lys, is a specific inhibitor of the trypsin-like enzyme family (tryptases) (Blow, D. M., Acc Chem Res, 1976, 9:145-152; Wilcox, P. E., Methods Enzymol, 1970, 19:64-108). The lack of protective effect of TLCK on the subsequent binding of FAM-VAD-FMK, FFCK or FLCK provides additional evidence that chemical reactivity of halomethyl ketone moiety alone does not play a significant role in observed affinity of these ligands to their respective binding sites.

Interestingly, there was no evidence of a significant number of cells that would have activated caspases only, without the activation of either the sites reactive with FFCK or FLCK. Such cells would appear on the bivariate distributions of the SR-VAD-FMK vs FFCK or FLCK (FIGS. 4 and 5) as the cells that have only red, with no green fluorescence; the vast majority of cells had components of both green and red fluorescence. This indicates that activation of caspases was rapidly followed by activation of the serpases and the time-window when only the former would be active, was relatively short.

The methodology of using affinity binding inhibitors to label the active enzymatic center (affinity-labeling of enzymatic center; ALEC) in situ has been introduced before, to detect active esterases in situ, in different tissues, (Ostrowski et al., (1963) Exp. Cell Res., 1963, 31:89-99), proteases (Darzynkiewicz et al., Nature, 1966, 212:1198-1203), or folate reductase (Darzynkiewicz et al., Science, 1966, 131:1538-1530) by radioisotope-labeled specific inhibitors of these enzymes. Application of FLICA to assay activation of caspases opens new possibilities to study these enzymes in living cells, detect their localization, and correlate the process of their activation with other events of apoptosis (Bedner et al., Exp Cell Res., 2000, 259:308-313; Smolewski et al., Cytometry, 2001, 44:73-82; Darzynkiewicz et al., Methods Mol Biol 2002, 203:289-299).

Recently, FLICA was applied in dual function, to arrest apoptosis and to label the cells arrested in apoptosis. This application allowed estimates of the kinetics of cell entry into apoptosis or cell death rate to be made (Smolewski et al., Int J Oncol, 2001, 19:657-663). As the present data indicate, based on the same principle, the in situ affinity labeling of enzyme active centers, FLISP offers a useful tool to investigate activation of Ser proteases. This tool will be particularly useful, because unlike caspases, little is known regarding particular Ser proteases, their mode of activation, intracellular distribution, and their preferred substrates. In addition to establishing the specificity of the FLISP reagents with respect to the caspase inhibitors, and with each other (described above), the affinity labels of the invention can also be used to determine the differences in activation of caspases compared to Ser proteases in different cell systems. These affinity labels can be used to study different models of apoptosis, and to differentiate between apoptosis and necrosis in a cell. The affinity labels of this invention also provide an opportunity to detect activation of these enzymes in situ, within the live cells, and thus to explore their localization and possible translocations. Based on a covalent 1:1 stoichiometry binding relationship to the active enzyme centers, these affinity labels also offer the means to quantify the respective enzymes within individual cells or cell organelles.

### EXAMPLE 2

### Synthesis of 5(6)-carboxyfluoresceinyl-L-phenylalanylchloromethyl ketone (FFCK)

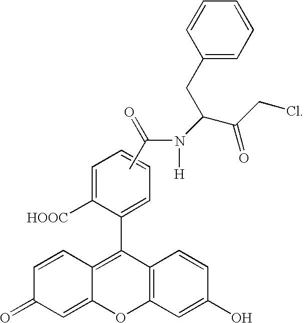 5-(and-6-)-Carboxyfluorescein, succinimidyl ester (80 mg, 0.17 mmole, FW 473.39, 5(6)-FAM) (Molecular Probes Inc., Eugene, Oreg., catalog number C-1311) was dissolved in 3 mL of dimethyl formamide (DMF). Phenylalanylchloromethyl ketone hydrochloride (40 mg, 0.17 mmole, FW 234) (Bachem Bioscience Inc., King of Prussia, Pa., catalog number N-1060) and diisopropylethyl amine (90 μL, Aldrich, Milwaukee, Wis.) were added to the solution. The reaction mixture was protected from light, stirred at room temperature for one hour and the solvent removed by rotary evaporation to provide an orange solid. The solid was partitioned between ethyl acetate and 10% aqueous hydrochloric acid (HCl), washed with 10% HCl and then water. The ethyl acetate fraction was dried over magnesium sulfate and the ethyl acetate removed by rotary evaporation to provide 35 mg dry weight, (37% yield) of 5(6)-carboxyfluoresceinyl-L-phenylalanylchloromethyl ketone (FFCK). Thin layer chromatography on silica gel (ethyl acetate: acetic acid, 97:3) gave a single spot of R_{f} 0.6.

### EXAMPLE 3

### Synthesis of 5(6)-carboxyfluorescyl-L-leucylchloromethyl ketone (FLCK)

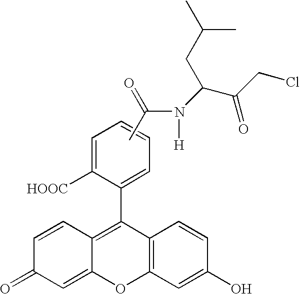

5-(and-6-)-Carboxyfluorescein, succinimidyl ester (82 mg, 0.17 mmole, FW 473.39, 5(6)-FAM) (Molecular Probes Inc., Eugene, Oreg., catalog number C-1311) was dissolved in 3 mL of dimethyl formamide (DMF). Leucylchloromethyl ketone ((35 mg, 0.17 mmole, FW 200.11) (Bachem Bioscience Inc., King of Prussia, Pa., catalog number N-1105) and diisopropylethyl amine (92 ul, Aldrich, Milwaukee, Wis.) were added to the solution. The reaction mixture was protected from light, stirred at room temperature for one hour and the solvent removed by rotary evaporation to provide an orange solid. The solid was partitioned between ethyl acetate and 15% aqueous hydrochloric acid (HCl), washed with 15% HCl and then water. The ethyl acetate fraction was dried over magnesium sulfate and the ethyl acetate removed by rotary evaporation to provide 72 mg dry weight, (81% yield) of 5(6)-carboxyfluoresceinyl-L-leucylchloromethyl ketone (FLCK). Thin layer chromatography on silica gel (ethyl acetate:acetic acid, 97:3) gave a single spot of R_{f} 0.7.

### EXAMPLE 4

Using procedures similar to those described herein, the following compounds of the formula (I) can also be prepared.

### 5(6)-Carboxyfluoresceinyl-L-lysylchloromethyl ketone

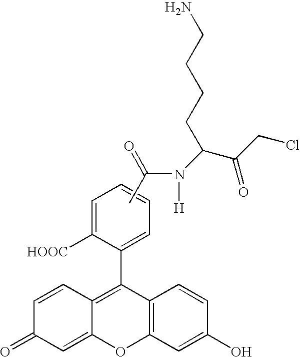

### 5(6)-Carboxyfluoresceinyl-L-arginylchloromethyl ketone

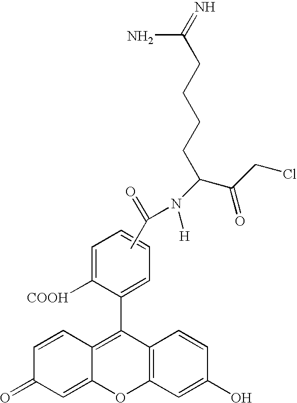

### Sulforhodaminyl-L-phenylalanylchloromethyl ketone

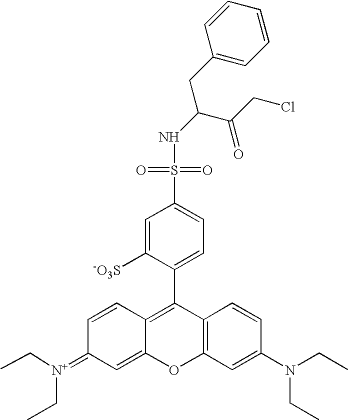

### Sulforhodaminyl-L-leucylchloromethyl ketone

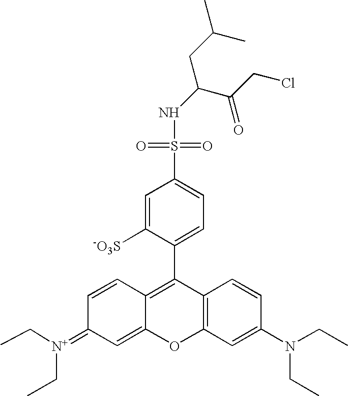

### Sulforhodarninyl-L-lysylchloromethyl ketone

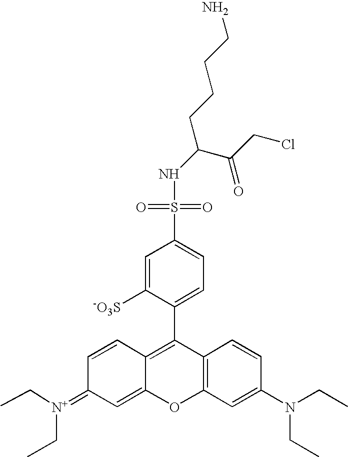

### Sulforhodaminyl-L-arginylchloromethyl ketone(I)

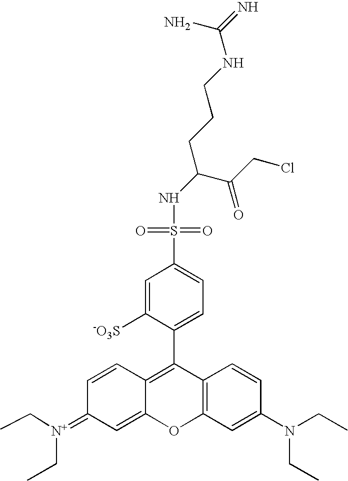

All publications, patents, and patent documents including 60/342,955, 60/342,778 and 60/342,704 are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for determining the apoptotic state of one or more viable whole cells, comprising: 1) contacting the cells with a caspase affinity labeling agent and with a serine protease affinity labeling agent; and 2) detecting the presence or abundance of each affinity labeling agent in the cells; wherein the presence or abundance of the caspase affinity labeling agent and the presence or abundance of the serine protease affinity labeling agent correlate with the apoptotic state of the cells.

2. The method of claim 1 wherein the cells are permeablized prior to contact with the agents.

3. The method of claim 1 wherein the presence of the caspase affinity labeling agent is detected concurrently with the detection of the presence of the serine protease affinity label.

4. The method of claim 1 wherein the presence of the caspase affinity labeling agent is detected before or after the detection of the presence of the serine protease affinity label.

5. The method of claim 1 wherein contacting the cells with the caspase affinity labeling agent is carried out concurrently with contacting the cells with the serine protease affinity labeling agent.

6. The method of claim 1 wherein the caspase affinity labeling agent is a red-labeled caspase affinity labeling agent and the serine protease affinity labeling agent is a green-labeled serine protease affinity labeling agent.

7. The method claim 1 wherein the caspase affinity labeling agent is a green-labeled caspase affinity labeling agent and the serine protease affinity labeling agent is a red-labeled serine protease affinity labeling agent.

8. The method of claim 1 wherein the caspase affinity labeling agent is a red-labeled caspase affinity labeling agent and the serine protease affinity labeling agent is a cold-labeled serine protease affinity labeling agent.

9. The method of claim 1 wherein the caspase affinity labeling agent is a green-labeled caspase affinity labeling agent and the serine protease affinity labeling agent is a cold-labeled serine protease affinity labeling agent.

10. The method of claim 1 wherein the caspase affinity labeling agent is a cold-labeled caspase affinity labeling agent and the serine protease affinity labeling agent is a green-labeled serine protease affinity labeling agent.

11. The method of claim 1 wherein the caspase affinity labeling agent is a cold-labeled caspase affinity labeling agent and the serine protease affinity labeling agent is a red-labeled serine protease affinity labeling agent.

12. The method of claim 1 wherein the caspase affinity labeling agent is a red-labeled caspase affinity labeling agent and the serine protease affinity labeling agent is a red-labeled serine protease affinity labeling agent.

13. The method of claim 1 wherein the caspase affinity labeling agent is a green-labeled caspase affinity labeling agent and the serine protease affinity labeling agent is a green-labeled serine protease affinity labeling agent.

14. The method of claim 1 wherein the caspase affinity labeling agent is a cold-labeled caspase affinity labeling agent, the serine protease affinity labeling agent is a cold-labeled serine protease affinity label and a green-labeled probe monitoring another cell process is introduced.

15. The method of claim 1 wherein the caspase affinity labeling agent is a cold-labeled caspase affinity labeling agent, the serine protease affinity labeling agent is a cold-labeled serine protease affinity label and a red-labeled probe monitoring another cell process is introduced.

16. The method of claim 1 wherein the caspase affinity labeling agent is a cold-labeled caspase affinity labeling agent, the serine protease affinity labeling agent is a cold-labeled serine protease affinity label and both a green-labeled probe and a red-labeled probe monitoring other cell processes are introduced.

17. The method of claim 1 wherein contacting the cells with the caspase affinity labeling agent is carried out before or after contacting the cells with the serine protease affinity labeling agent.

18. The method of claim 1 wherein the caspase affinity labeling agent is a compound of formula II:
L₁-A₁-X₁—NH—CH(R₁′)C(═O)CH₂F (II)
wherein:
L₁ is a detectable group;
A₁ is a direct bond or a linker;
X₁ is absent, an amino acid, or a peptide; and
R₁′ must be the aspartic acid side-chain (CH₂—COOH) or an ester of aspartic acid (—CH₂CO₂R, where R is CH₃, C₂H₅ or CH₂C₆H₅) as example.

19. The method of claim 1 wherein the caspase affinity labeling agent contains the free aspartic acid (D) or methyl ester (D-O—CH₃) of aspartic acid within the 5(6)-carboxyfluoresceinyl-L-valylalanylaspartylfluoromethyl ketone (FAM-VAD-FMK) or sulforhodaminyl-L-valylalanylaspartylfluoromethyl ketone (SR-VAD-FMK); or a salt thereof.

20. The method of claim 1 wherein the serine protease affinity labeling agent is a compound of formula I:
L-A-X—NH—CH(R′)C(═O)CH₂Cl (I)
wherein:
L is a detectable group;
A is a direct bond or a linker;
X is absent, an amino acid, or a peptide;
R′ is hydrogen or (C₁-C₆)alkyl, wherein the alkyl is optionally substituted with one or more (1, 2, 3, or 4) substituents independently selected from the group consisting of guanidino, —C(═O)NRₐR_{b}, —C(═O)OR_{c}, halo, —NRₐR_{b}, aryl; heteroaryl, —OR_{c}, or —SR_{c};
each Rₐ and R_{b} is independently hydrogen, (C₁-C₆)alkyl, phenyl, benzyl, or phenethyl; or Rₐ and R_{b} together with the nitrogen to which they are attached form a pyrrolidino, morpholino, or thiomorpholino ring; and
each R_{c} is independently hydrogen, (C₁-C₆)alkyl, phenyl, benzyl, or phenethyl;
wherein any aryl or heteroaryl is optionally substituted with one or more (e.g. 1, 2, 3, or 4) substituents independently, selected from the group consisting of halo, nitro, cyano, hydroxy, mercapto, (C₁-C₆)alkyl, (C₁-C₆)alkoxy, trifluoromethyl, or trifluoromethoxy;
or a salt thereof.

21. The method of claim 18 wherein the serine protease affinity labeling agent is a compound of formula I as described in claim 20; or a salt thereof.

22. The method of claim 1 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

23. The method of claim 18 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

24. A diagnostic method for determining the presence or absence of a disease characterized by the presence of one or more active serine proteases and the presence of one or more caspases in one or more viable whole cells, comprising: 1) contacting the cells with a caspase affinity labeling agent and a serine protease affinity labeling agent; and 2) detecting the presence or relative abundance of each affinity labeling agent in the cells; wherein the presence or relative abundance of the caspase affinity labeling agent and the presence or relative abundance of the serine protease affinity labeling agent correlate with the presence or absence of the disease.

25. The method of claim 24 wherein the cells are permeablized prior to contact with the agents.

26. The method of claim 24 wherein the presence of the caspase affinity labeling agent is detected concurrently with the detection of the presence of the serine protease affinity label.

27. The method of claim 24 wherein the presence of the caspase affinity labeling agent is detected before or after the detection of the presence of the serine protease affinity label.

28. The method of claim 24 wherein contacting the cells with the caspase affinity labeling agent is carried out concurrently with contacting the cells with the serine protease affinity labeling agent.

29. The method of claim 24 wherein contacting the cells with the caspase affinity labeling agent is carried out before or after contacting the cells with the serine protease affinity labeling agent.

30. The method of claim 24 wherein the caspase affinity labeling agent is a compound of formula II as described in claim 18; or a salt thereof.

31. The method of claim 24 wherein the caspase affinity labeling agent contains the free aspartic acid (D) or methyl ester (D-O—CH₃) of aspartic acid within the 5(6)-carboxyfluoresceinyl-L-valylalanylaspartylfluoromethyl ketone (FAM-VAD-FMK) or sulforhodaminyl-L-valylalanylaspartylfluoromethyl ketone (SR-VAD-FMK); or a salt thereof.

32. The method of claim 24 wherein the serine protease affinity labeling agent is a compound of formula I as described in claim 20; or a salt thereof.

33. The method of claim 30 wherein the serine protease affinity labeling agent is a compound of formula I as described in claim 20; or a salt thereof.

34. The method of claim 24 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

35. The method of claim 30 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

36. A method for determining whether a therapeutic agent induces apoptosis in one or more viable whole cells, comprising: 1) contacting the cells with the therapeutic agent; 2) contacting the cells with a caspase affinity labeling agent and a serine protease affinity labeling agent; and 3) detecting the presence or abundance of each of the affinity labeling agents in the cells; wherein the presence or relative abundance of the caspase affinity labeling agent and the presence or relative abundance of the serine protease affinity labeling agent correlate with the ability of the agent to induce apoptosis.

37. The method of claim 36 wherein the cells are contacted with the therapeutic agent before the cells are contacted with the affinity labeling agents.

38. The method of claim 36 wherein the cells are contacted with the therapeutic agent at the same time the cells is contacted with the affinity labeling agents.

39. The method of claim 36 wherein the therapeutic agent is an anti-cancer agent (used to induce apoptosis in cancer cells) consisting of, but not be limited to; 1) DNA cleavage reagents, 2) anti-metabolites, 3) mitotic inhibitors, 4) nucleotide analogs, 5) topoisomerase inhibitors, and 6) as well as other intracellular mechanistic targeting molecules in use today and to be developed in the future.

40. The method of claim 36 wherein the therapeutic agent is a topoisomerase inhibitor (which induces apoptosis by causing errors in DNA replication), comprised of; 1) type I topoisomerase inhibitor, 2) type II topoisomerase inhibitor, or 3) other topoisomerase type inhibitors in use today and to be developed in the future.

41. The method of claim 36 wherein the cells are permeablized prior to contact with the affinity labeling agents.

42. The method of claim 36 wherein the presence of the caspase affinity labeling agent is detected concurrently with the detection of the presence of the serine protease affinity label.

43. The method of claim 36 wherein the presence of the caspase affinity labeling agent is detected before or after the detection of the presence of the serine protease affinity label.

44. The method of claim 36 wherein contacting the cells with the caspase affinity labeling agent is carried out concurrently with contacting the cells with the serine protease affinity labeling agent.

45. The method of claim 36 wherein contacting the cells with the caspase affinity labeling agent is carried out before or after contacting the cells with the serine protease affinity labeling agent.

46. The method of claim 36 wherein the caspase affinity labeling agent is a compound of formula II as described in claim 7; or a salt thereof.

47. The method of claim 36 wherein the caspase affinity labeling agent contains the free aspartic acid (D) or methyl ester (D-O—CH₃) of aspartic acid within the 5(6)-carboxyfluoresceinyl-L-valylalanylaspartylfluoromethyl ketone (FAM-VAD-FMK) or sulforhodaminyl-L-valylalanylaspartylfluoromethyl ketone (SR-VAD-FMK); or a salt thereof.

48. The method of claim 36 wherein the serine protease affinity labeling agent is a compound of formula I as described in claim 9; or a salt thereof.

49. The method of claim 46 wherein the serine protease affinity labeling agent is a compound of formula I as described in claim 9; or a salt thereof.

50. The method of claim 46 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

51. The method of claim 46 wherein the serine protease affinity labeling agent is 5(6)-carboxyfluoresceinyl-L-phenylalanylchloromethyl ketone, 5(6)-carboxyfluoresceinyl-L-leucylchloromethyl ketone, 5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, sulforhodaminyl-L-lysylchloromethyl ketone, sulforhodaminyl-L-arginylchloromethyl ketone; or a salt thereof.

52-82. (canceled)

83. A diagnostic method for determining the presence of a tumor in a tissue sample comprising: 1) contacting the sample with a caspase affinity labeling agent and a serine protease affinity labeling agent; and 2) detecting the presence or abundance of each of the affinity labeling agents in the cells; wherein the presence or abundance of the caspase affinity labeling agent and the presence or abundance of the serine protease affinity labeling agent correlate with the presence of a tumor.

84-88. (canceled)


SEQUENCE LISTING (12 sequences)

SEQ ID NO: 1 - protein, 4 aa, Artificial Sequence
  A synthetic peptide
YVAD

SEQ ID NO: 2 - protein, 4 aa, Artificial Sequence
  A synthetic peptide
WEHD

SEQ ID NO: 3 - protein, 5 aa, Artificial Sequence
  A synthetic peptide
VDVAD

SEQ ID NO: 4 - protein, 4 aa, Artificial Sequence
  A synthetic peptide
DEHD

SEQ ID NO: 5 - protein, 4 aa, Artificial Sequence
  A synthetic peptide
DEVD

SEQ ID NO: 6 - protein, 4 aa, Artificial Sequence
  A synthetic peptide
XEHD

SEQ ID NO: 7 - protein, 4 aa, Artificial Sequence
  A synthetic peptide
LEHD

SEQ ID NO: 8 - protein, 4 aa, Artificial Sequence
  A synthetic peptide
VEID

SEQ ID NO: 9 - protein, 4 aa, Artificial Sequence
  A synthetic peptide
LETD

SEQ ID NO: 10 - protein, 4 aa, Artificial Sequence
  A synthetic peptide
AEVD

SEQ ID NO: 11 - protein, 4 aa, Artificial Sequence
  A synthetic peptide
LELD

SEQ ID NO: 12 - protein, 4 aa, Artificial Sequence
  A synthetic peptide
LEED